(19) United States
(12) Patent Application Publication
(10) Pub. No.: US 2007/0128270 A1
(43) Pub. Date: Jun. 7, 2007

(54) PHARMACEUTICAL FORMULATIONS CONTAINING 5-CYCLOPROPYL-2-(4-FLUOROPHENYL)-6-[(2-HYDROXYETHYL)(METHYLSULFONYL)AMINO]-N-METHYL-1-BENZOFURAN-3-CARBOXAMIDE AND METHOD OF MAKING THE SAME

(75) Inventors: Mannching Sherry Ku, Thiells, NY (US); Weiyi Li, Pompton Plains, NJ (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/558,388

(22) Filed: Nov. 9, 2006

Related U.S. Application Data
(60) Provisional application No. 60/735,191, filed on Nov. 10, 2005.

(51) Int. Cl. A61K 31/34; A61K 9/48; A61K 9/20

(52) U.S. Cl. 424/451; 514/471; 424/464

(57) ABSTRACT

Pharmaceutical formulations containing 5-cyclopropyl-2-(4-fluorophenyl)-6-[(2-hydroxyethyl)(methylsulfonyl)amino]-N-methyl-1-benzofuran-3-carboxamide and pharmaceutically acceptable additives including at least one surfactant are made.

This application claims the benefit of U.S. Provisional Patent Application No. 60/735,191, filed Nov. 10, 2005, the entire disclosure of which is incorporated by reference herein.

### BACKGROUND OF THE INVENTION

1. Field of the Invention

The present invention is directed to pharmaceutical formulations containing 5-cyclopropyl-2-(4-fluorophenyl)-6-[(2-hydroxyethyl)(methylsulfonyl)amino]-N-methyl-1-benzofuran-3-carboxamide, as well as to methods of making such pharmaceutical formulations and a method of treating a subject with such pharmaceutical formulations.

2. Related Background Art

The hepatitis C virus inhibitor 5-cyclopropyl-2-(4-fluorophenyl)-6-[(2-hydroxyethyl)(methylsulfonyl)amino]-N-methyl-1-benzofuran-3-carboxamide is a potent inhibitor of the hepatitis C virus and has shown very favorable toxicological and pharmacological profiles. The structure of 5-cyclopropyl-2-(4-fluorophenyl)-6-[(2-hydroxyethyl)(methylsulfonyl)amino]-N-methyl-1-benzofuran-3-carboxamide is as follows:

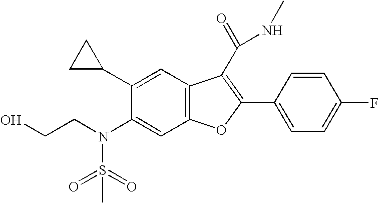

However, formulating 5-cyclopropyl-2-(4-fluorophenyl)-6-[(2-hydroxyethyl)(methylsulfonyl)amino]-N-methyl-1-benzofuran-3-carboxamide for oral dosage has proven very difficult, as 5-cyclopropyl-2-(4-fluorophenyl)-6-[(2-hydroxyethyl)(methylsulfonyl)amino]-N-methyl-1-benzofuran-3-carboxamide is insoluble in aqueous medium at gastrointestinal pHs. Accordingly, there is a need to develop an oral dosage form containing 5-cyclopropyl-2-(4-fluorophenyl)-6-[(2-hydroxyethyl)(methylsulfonyl)amino]-N-methyl-1-benzofuran-3-carboxamide, which has good bioavailability properties and which can be produced according to a reliable and robust process.

### SUMMARY OF THE INVENTION

In a first aspect, the present invention is directed to a pharmaceutical formulation comprising a therapeutically effective amount of 5-cyclopropyl-2-(4-fluorophenyl)-6-[(2-hydroxyethyl)(methylsulfonyl)amino]-N-methyl-1-benzofuran-3-carboxamide and pharmaceutically acceptable additives, wherein said pharmaceutically acceptable additives comprise at least one surfactant. In a particularly preferred embodiment, the pharmaceutically acceptable additives further comprise at least one solubilizer.

In a second aspect, the present invention is directed to a method of making a pharmaceutical formulation comprising the steps of (a) granulating 5-cyclopropyl-2-(4-fluorophenyl)-6-[(2-hydroxyethyl)(methylsulfonyl)amino]-N-methyl-1-benzofuran-3-carboxamide and pharmaceutically acceptable additives to form a granulate, wherein said pharmaceutically acceptable additives comprise at least one surfactant; and (b) blending the granulate with pharmaceutically acceptable additives to form a final blend. Optionally, the inventive method further comprises the step of (c) encapsulating the final blend to form the pharmaceutical formulation or (c) compressing the final blend to form the pharmaceutical formulation. In a particularly preferred embodiment, the pharmaceutically acceptable additives in step (a) further comprise at least one solubilizer.

In preferred embodiments of the inventive method step (a) comprises the steps of (a1) screening 5-cyclopropyl-2-(4-fluorophenyl)-6-[(2-hydroxyethyl)(methylsulfonyl)amino]-N-methyl-1-benzofuran-3-carboxamide, at least one diluent, at least one solubilizer, at least one disintegrant, and at least one surfactant into a granulator to form a screened material; (a2) blending the screened material to form a screened/blended material; (a3) dissolving at least one surfactant in water to form a surfactant solution; (a4) granulating the screened/blended material with the surfactant solution to form a wet granulation; (a5) drying the wet granulation to form a dried granulation; and (a6) milling the dried granulation to form the granulate. In still other preferred embodiments, step (b) comprises the steps of (b1) blending at least one screened glidant with the granulate from step (a) to form a first blend; (b2) blending the first blend with at least one screened solubilizer and at least one screened disintegrant to form a second blend; (b3) blending a portion of the second blend with an equal amount of at least one screened lubricant to form a third blend; and (b4) blending the third blend with the remaining second blend to form the final blend.

In a third aspect, the present invention is directed to a pharmaceutical formulation made according to the inventive method.

In a fourth aspect, the present invention is directed to a method of inhibiting hepatitis C virus, wherein the method comprises administering a pharmaceutical formulation of the present invention to a subject in need of such treatment.

In certain preferred embodiments of this invention, the therapeutically effective amount of 5-cyclopropyl-2-(4-fluorophenyl)-6-[(2-hydroxyethyl)(methylsulfonyl)amino]-N-methyl-1-benzofuran-3-carboxamide ranges from about 1 mg to about 2000 mg, more preferably from about 10 mg to about 400 mg and most preferably from about 25 mg to about 200 mg, and/or the 5-cyclopropyl-2-(4-fluorophenyl)-6-[(2-hydroxyethyl)(methylsulfonyl)amino]-N-methyl-1-benzofuran-3-carboxamide is micronized to a particle size specification of 50% less than or equal to 5 μm and 90% less than or equal to 20 μm. In other preferred embodiments, the at least one surfactant is a blend of surfactants, more preferably a blend of sodium lauryl sulfate and polysorbate 80; in still other preferred embodiments, the at least one solubilizer is povidone. In still other preferred embodiments, the pharmaceutically acceptable additives further comprise ingredients selected from the group consisting of diluents, surfactants, solubilizers, disintegrants, glidants, lubricants, colorants and combinations thereof.

### BRIEF DESCRIPTION OF THE DRAWINGS

FIG. 1 shows the mean (SD) plasma 5-cyclopropyl-2-(4-fluorophenyl)-6-[(2-hydroxyethyl)(methylsulfonyl)amino]-N-methyl-1-benzofuran-3-carboxamide levels in beagle dogs (n=4) following single oral dose of a 150 mg tablet made according to the present invention.

FIG. 2 shows the mean (SD) plasma 5-cyclopropyl-2-(4-fluorophenyl)-6-[(2-hydroxyethyl)(methylsulfonyl)amino]-N-methyl-1-benzofuran-3-carboxamide levels in beagle dogs comparing tablet and capsule pharmaceutical formulations of the present invention.

### DETAILED DESCRIPTION

The first embodiment of the invention is directed to a pharmaceutical formulation comprising a therapeutically effective amount of 5-cyclopropyl-2-(4-fluorophenyl)-6-[(2-hydroxyethyl)(methylsulfonyl)amino]-N-methyl-1-benzofuran-3-carboxamide and pharmaceutically acceptable additives. The pharmaceutically acceptable additives of the first embodiment necessarily comprise at least one surfactant to effect fast and complete dissolution of 5-cyclopropyl-2-(4-fluorophenyl)-6-[(2-hydroxyethyl)(methylsulfonyl)amino]-N-methyl-1-benzofuran-3-carboxamide, given its insolubility in aqueous medium at gastrointestinal pHs.

In fact, according to a preferred embodiment, the at least one surfactant is a blend of surfactants, more preferably a blend of sodium lauryl sulfate and polysorbate 80 (Tween 80). In a particularly preferred embodiment, the pharmaceutically acceptable additives further comprise at least one solubilizer. Preferably the solubilizer is povidone. Hence a pharmaceutical formulation of 5-cyclopropyl-2-(4-fluorophenyl)-6-[(2-hydroxyethyl)(methylsulfonyl)amino]-N-methyl-1-benzofuran-3-carboxamide with the combination of sodium lauryl sulfate, polysorbate 80 and povidone is a preferred embodiment of this invention; the present inventors have found that this combination is effective in achieving fast and complete dissolution of 5-cyclopropyl-2-(4-fluorophenyl)-6-[(2-hydroxyethyl)(methylsulfonyl)amino]-N-methyl-1-benzofuran-3-carboxamide. Solubility of 5-cyclopropyl-2-(4-fluorophenyl)-6-[(2-hydroxyethyl)(methylsulfonyl)amino]-N-methyl-1-benzofuran-3-carboxamide is improved from 0.02 mg/mL to 0.64, 0.16 and 0.14 mg/mL in 2% sodium lauryl sulfate, 10% povidone, and 2% polysorbate 80, respectively.

In more preferred embodiments, the sodium lauryl sulfate is present in an amount ranging from about 1% to about 10%, more preferably from about 4% to about 6%, and most preferably is about 5%, by weight of the pharmaceutical formulation. In more preferred embodiments, the polysorbate 80 is present in an amount ranging from about 1% to about 5%, more preferably from about 2% to about 4%, and most preferably is about 3%, by weight of the pharmaceutical formulation. In more preferred embodiments, the povidone is present in an amount ranging from about 1% to about 20%, more preferably from about 8% to about 12%, and most preferably is about 10%, by weight of the pharmaceutical formulation. In preferred embodiments of this invention, the 5-cyclopropyl-2-(4-fluorophenyl)-6-[(2-hydroxyethyl)(methylsulfonyl)amino]-N-methyl-1-benzofuran-3-carboxamide is present in an amount of at least about 60% by weight of the pharmaceutical formulation.

5-Cyclopropyl-2-(4-fluorophenyl)-6-[(2-hydroxyethyl)(methylsulfonyl)amino]-N-methyl-1-benzofuran-3-carboxamide suitable for use in the present invention can be prepared as previously described in U.S. Patent Application Publication No. 2004-0162318. 5-Cyclopropyl-2-(4-fluorophenyl)-6-[(2-hydroxyethyl)(methylsulfonyl)amino]-N-methyl-1-benzofuran-3-carboxamide may be used for purposes of this invention in any of its amorphous, crystalline, hydrated or solvated forms. Polymorphic forms of 5-cyclopropyl-2-(4-fluorophenyl)-6-[(2-hydroxyethyl)(methylsulfonyl)amino]-N-methyl-1-benzofuran-3-carboxamide and methods of making the same are disclosed in co-pending U.S. patent application Ser. No. ______ (based on U.S. Provisional Application No. 60/735,190, which is incorporated by reference herein. In a preferred embodiment of this invention, the 5-cyclopropyl-2-(4-fluorophenyl)-6-[(2-hydroxyethyl)(methylsulfonyl)amino]-N-methyl-1-benzofuran-3-carboxamide is micronized to a particle size specification of 50% less than or equal to 5 μm and 90% less than or equal to 20 μm.

5-Cyclopropyl-2-(4-fluorophenyl)-6-[(2-hydroxyethyl)(methylsulfonyl)amino]-N-methyl-1-benzofuran-3-carboxamide is employed in a therapeutically effective amount. A “therapeutically effective amount” is intended to mean the amount of 5-cyclopropyl-2-(4-fluorophenyl)-6-[(2-hydroxyethyl)(methylsulfonyl)amino]-N-methyl-1-benzofuran-3-carboxamide that, when administered to a subject in need thereof, is sufficient to effect treatment for disease conditions alleviated by the inhibition of hepatitis C virus. The amount of a given compound of the invention that will be therapeutically effective will vary depending upon factors such as the disease condition and the severity thereof, the identity of the subject in need thereof, etc., which amount may be routinely determined by artisans of ordinary skill in the art. Typically, the therapeutically effective amount ranges from about 1 mg to about 2000 mg, more preferably from about 10 mg to about 400 mg, and most preferably from about 25 mg to about 200 mg.

Pharmaceutically acceptable additives suitable for use in the present invention include, without limitation, diluents, surfactants, solubilizers, disintegrants, glidants, lubricants, colorants and combinations thereof.

Suitable diluents include, without limitation, microcrystalline cellulose, silicified microcrystalline cellulose, starches, mannitol, lactose, celluloses, calcium phosphates and combinations thereof. When present, a diluent may be employed in an amount ranging from about 10% to about 80%, preferably from about 15% to about 70%, and more preferably is about 16% or about 66% by weight of the pharmaceutical formulation.

Suitable surfactants include, without limitation, polysorbate 80, sodium lauryl sulfate, sugar esters of fatty acids, poloxamer, docusate sodium, polyoxyethylene sorbitan fatty acid esters, and combinations thereof. The surfactant or mixture of surfactants is employed in an amount ranging from about 2% to about 15%, preferably from about 6% to about 10% and more preferably is about 8% by weight of the pharmaceutical formulation.

Suitable solubilizers include, without limitation, povidone, poloxamer, glycerides of fatty acids, polyoxyethylene castor oil derivatives, and combinations thereof. When present, a solubilizer may be employed in an amount ranging from about 1% to about 20%, preferably from about 8% to about 12%, and more preferably is about 10% by weight of the pharmaceutical formulation.

Suitable disintegrants include, without limitation, sodium starch glycolate, crospovidone, croscarmellose sodium, alginic acid, modified cellulose, pregelatinized starch, ion exchange resins, and combinations thereof. When present, a disintegrant may be employed in an amount ranging from about 1% to about 10%, preferably from about 4% to about 6%, and more preferably is about 5% by weight of the pharmaceutical formulation.

Suitable glidants include, without limitation, colloidal silicon dioxide, talc, metal stearates, magnesium carbonate, calcium silicate, fumed silicon dioxide, and combinations thereof. When present, a glidant may be employed in an amount ranging from about 0.1% to about 1%, preferably from about 0.1% to about 0.3%, and more preferably is about 0.2% by weight of the pharmaceutical formulation.

Suitable lubricants include, without limitation, magnesium stearate, other metal stearates, glyceryl behenate, sodium stearyl fumarate, hydrogenated vegetable oils, fatty acids, and combinations thereof. When present, a lubricant may be employed in an amount ranging from about 0.2% to about 2%, preferably from about 0.4% to about 0.6%, and more preferably is about 0.5% by weight of the pharmaceutical formulation.

Suitable colorants include, without limitation, FD&C approved colorants or combinations thereof. When present, a colorant may be employed in an amount readily determinable by one of ordinary skill in the art.

In one preferred embodiment, the pharmaceutical formulation takes the form of granulated 5-cyclopropyl-2-(4-fluorophenyl)-6-[(2-hydroxyethyl)(methylsulfonyl)amino]-N-methyl-1-benzofuran-3-carboxamide in a capsule. In an additional preferred embodiment, the pharmaceutical formulation takes the form of granulated and compressed 5-cyclopropyl-2-(4-fluorophenyl)-6-[(2-hydroxyethyl)(methylsulfonyl)amino]-N-methyl-1-benzofuran-3-carboxamide, i.e., the form of a tablet. Granulation may be accomplished by the method of the second embodiment of the invention (see below) or by any other suitable means. Any suitable capsule of any suitable size may be used; typically, the capsule is a hydroxypropyl methylcellulose, hypromellose or gelatin capsule, though the capsule is not limited thereto. Compression or tabletting may be accomplished by any convention compression or tabletting means or method; tablets of any suitable size or shape are possible.

The second embodiment of the present invention is directed to a method of making a pharmaceutical formulation comprising the steps of (a) granulating 5-cyclopropyl-2-(4-fluorophenyl)-6-[(2-hydroxyethyl)(methylsulfonyl)amino]-N-methyl-1-benzofuran-3-carboxamide and pharmaceutically acceptable additives to form a granulate, wherein said pharmaceutically acceptable additives comprise at least one surfactant; and (b) blending the granulate with pharmaceutically acceptable additives to form a final blend. In a particularly preferred embodiment of the invention, the pharmaceutically acceptable additives of step (a) further comprise at least one solubilizer. Optionally the inventive method comprises the step of (c) encapsulating the final blend to form the pharmaceutical formulation in the form of a capsule or the step of (c) compressing the final blend to form the pharmaceutical formulation in the form of a tablet. All details regarding ingredient identities, amounts, etc. are the same as noted above with regard to the first embodiment of the invention.

Preferably, step (a) comprises a wet granulation process. More preferably, step (a) comprises the steps of (a1) screening 5-cyclopropyl-2-(4-fluorophenyl)-6-[(2-hydroxyethyl)(methylsulfonyl)amino]-N-methyl-1-benzofuran-3-carboxamide, at least one diluent, at least one solubilizer, at least one disintegrant, and at least one surfactant into a granulator to form a screened material; (a2) blending the screened material to form a screened/blended material; (a3) dissolving at least one surfactant in water to form a surfactant solution; (a4) granulating the screened/blended material with the surfactant solution to form a wet granulation; (a5) drying the wet granulation to form a dried granulation; and (a6) milling the dried granulation to form the granulate. Optionally step (a) further comprises the step of (a4*) adding additional water to facilitate granulation.

In step (a1), the 5-cyclopropyl-2-(4-fluorophenyl)-6-[(2-hydroxyethyl)(methylsulfonyl)amino]-N-methyl-1-benzofuran-3-carboxamide, at least one diluent, at least one solubilizer, at least one disintegrant, and at least one surfactant are screened (sieved, milled, etc.) into a granulator. Screening can be accomplished using any suitable means. Likewise, the granulator can be any suitable equipment. Typically the screened material is sieved through a 20 mesh sieve. It is important to note that, prior to step (a1), it is preferable to micronize the 5-cyclopropyl-2-(4-fluorophenyl)-6-[(2-hydroxyethyl)(methylsulfonyl)amino]-N-methyl-1-benzofuran-3-carboxamide to a particle size specification of 50% less than or equal to 5 μm and 90% less than or equal to 20 μm. In a preferred embodiment, the at least one surfactant in step (a1) is sodium lauryl sulfate and the at least one solubilizer is povidone.

In step (a2), the screened material is blended in a granulator to form a screened/blended material. Blending can be accomplished using any suitable means.

In steps (a3) and (a4), at least one surfactant is dissolved in water to form a surfactant solution, and the screened/blended material is blended with the surfactant solution to form a wet granulation. A surfactant solution is employed in the present inventive method in order to carry out a wet granulation process. A wet granulation process is believed necessary to accommodate the particle size of the ingredients and to improve powder flowability and density. Here again blending may be accomplished using any suitable means. In a preferred embodiment, the at least one surfactant is step (a3) is polysorbate 80. In an optional step (a4*), additional water may be added during blending to facilitate granulation.

In step (a5), the wet granulation is dried. Drying may be accomplished using any suitable means such as a fluid bed dryer at about 50° C. and is carried out until a loss on drying ranging from about 1% to about 4% is achieved.

In step (a6), the dried granulation is milled to form the granulate. Milling can be accomplished using any suitable means. Typically the dry granulation is milled through a screening mill with a screen size of about 0.0394 inches.

Alternatively, step (a), i.e., the provision of a granulate, can be accomplished by any known granulation technique which results in a granulate having the desired properties of density and flowability.

Further preferably, step (b) comprises the steps of (b1) blending at least one screened glidant with the granulate from step (a) to form a first blend; (b2) blending the first blend with at least one screened solubilizer and at least one screened disintegrant to form a second blend; (b3) blending a portion of the second blend with an equal amount of at least one screened lubricant to form a third blend; and (b4) blending the third blend with the remaining second blend to form the final blend.

In step (b1), at least one screened glidant is blended with the granulate from step (a) to form a first blend. First, at least one glidant is screened using any suitable means. Typically a 20 mesh sieve is used. Then the screened glidant is blended with the granulate from step (a). Blending can be accomplished using any suitable means.

In step (b2), the first blend is blended with at least one screened solubilizer and at least one screened disintegrant to form a second blend. First, at least one solubilizer and at least one disintegrant are screened using any suitable means. Typically a 20 mesh sieve is used. Then the screened solubilizer and disintegrant are blended with the first blend from step (b1). Blending can be accomplished using any suitable means.

In step (b3), an equal portion of the second blend and an equal amount of at least one screened lubricant are blended to form a third blend. First, at least one lubricant is screened using any suitable means. Typically a 20 mesh sieve is used. Then the screened lubricant is blended with a portion of the second blend from step (b2) in equal amounts. Blending can be accomplished using any suitable means.

In step (b4), the third blend from step (b3) is blended with the remaining second blend from step (b3) to form the final blend. Blending can be accomplished using any suitable means.

Alternatively, step (b), i.e., the provision of a final blend, can be accomplished by any known blending technique which results in a final blend having the desired properties.

Optional step (c) of the present inventive method may entail encapsulating the final blend of step (b) to form the pharmaceutical formulation. Encapsulation is accomplished by any suitable means, i.e., an encapsulation device. Likewise any suitable capsule may be used; typically, the capsule is of any suitable size and is a hydroxypropyl methylcellulose, hypromellose or gelatin capsule, though the capsule is not limited thereto. In a preferred embodiment of the present invention, a #0E sized capsule is used and filled to a target fill weight ranging from about 50 mg to about 500 mg.

Alternative step (c) of the present inventive method may entail compressing the final blend to form the pharmaceutical formulation in the form of a tablet. Compression or tabletting can be accomplished by any suitable means.

A third embodiment of the present invention is directed to a pharmaceutical formulation made according to the method of the second embodiment.

The fourth embodiment of the present invention is directed to a method of inhibiting hepatitis C virus, wherein the method comprises administering a pharmaceutical formulation as defined by the first or third embodiment of this invention to a subject in need of such treatment. In a preferred embodiment, the pharmaceutical formulation is orally administered to the subject.

Specific embodiments of the invention will now be demonstrated by reference to the following examples. It should be understood that these examples are disclosed solely by way of illustrating the invention and should not be taken in any way to limit the scope of the present invention.

### EXAMPLE 1

### 25 MG Capsule

A 2.5 kg batch of a pharmaceutical formulation of 5-cyclopropyl-2-(4-fluorophenyl)-6-[(2-hydroxyethyl)(methylsulfonyl)amino]-N-methyl-1-benzofuran-3-carboxamide was made as follows:
1. Weigh the following ingredients—micronized 5-cyclopropyl-2-(4-fluorophenyl)-6-[(2-hydroxyethyl)(methylsulfonyl)amino]-N-methyl-1-benzofuran-3-carboxamide (250.0 g), microcrystalline cellulose (Avicel PH101)(1657.5 g), povidone (USP Plasdone K29/32) (100 g), sodium starch glycolate (NF)(75 g) and sodium lauryl sulfate (NF)(125 g).
2. Screen the ingredients from step 1 through a 20 mesh screen.
3. Add half of the microcrystalline cellulose into a suitable granulator. Then add micronized 5-cyclopropyl-2-(4-fluorophenyl)-6-[(2-hydroxyethyl)(methylsulfonyl)amino]-N-methyl-1-benzofuran-3-carboxamide, povidone, sodium starch glycolate and sodium lauryl sulfate into the granulator.
4. Add the other half of the microcrystalline cellulose into the granulator. Mix for 2 minutes with impeller on approximately 350 rpm. Determine density (approximately 0.4 g/ml).
5. Add 75 g polysorbate 80 to 300 g purified water with stirring for a minimum of 30 minutes (at low speed to minimize foam formation). Visually verify complete dissolution of the polysorbate 80.
6. Granulate the mix in step 4 with the solution from step 5 with impeller on at low speed and chopper off.
7. Add additional purified water if necessary and mix until granulation is complete.
8. After all granulating fluid is added, mix for an additional 1 minute with impeller on and chopper off. Record total amount of water used to granulate.
9. Dry the granulation in a fluid bed dryer with an inlet temperature of 50° C. ±5° C. to a moisture content of about 1% to about 4% tested on a Computrac at 100° C. Note: Grind the granulation in a mortar before performing the moisture test. Weigh and record yield.
10. If two or more sub-batches of the granulation are made, add the sub-batches into a tumble type blender and blend for 5 minutes.
11. Perform a sieve analysis on the dried granulation with 42 mesh (0.0139 inch), 80 mesh (0.0070 inch), 150 mesh (0.0041 inch), 200 mesh (0.0029 inch), 325 mesh (0.0017 inch) and 400 mesh (0.0015 inch) sieves.
12. Pass the granulation through a cone mill approximately at 1000 rpm with 0.0394 inch round hole sieves.
13. Transfer the milled dried granulation from step 12 into a suitable tumble type blender (blending will be done without intensifier bar activation). Blend for 2 minutes. Note: Determine density (approximately 0.5 g/ml) and sieve analysis with same set of sieves. Take a 20 g formulator sample. Weigh and record yield. Review formulator particle size result before proceeding.
14. Store in a black double polyethylene bag in an appropriate container at room temperature until ready for final blend. Protect from light.
15. Based on the yield in step 13, calculate the amounts required for the dry addition. Theoretical amounts for a 2.5 kg batch are given as: silicon dioxide (colloidal, NF)(5 g), povidone (USP Plasdone K 29/32)(150 g), sodium starch glycolate (NF)(50 g) and magnesium stearate (NF/EP, vegetable grade)(12.5 g).
16. Transfer the milled dried granulation from step 14 into a tumble type blender.
17. Weigh silicon dioxide and pass through a 20 mesh screen and add into the tumble type blender. Blend for 5 minutes.
18. Weigh the povidone, sodium starch glycolate and pass through the 20 mesh screen and add into the tumble type dryer. Blend for 10 minutes.
19. Pass magnesium stearate through a 30 mesh screen and pre-mix with an approximately equal portion of blend (may bag blend for 15 sec), then add to the blend in the tumble type blender. Blend for 2 minutes. Note: Determine density (approximately 0.4 g/ml) and sieve analysis. Take 12 samples for blend uniformity (approximately 350 mg) and a 50 g formulator sample. Weigh and record yield. Store in a black double polyethylene bag in an appropriate container at room temperature until ready for encapsulation. Protect from light.
20. Set up a H&K capsule machine with parts for size #0E capsules (suggested dosing disk: 12 mm) and tamping pins for size #0E capsules.
21. Encapsulate the 5-cyclopropyl-2-(4-fluorophenyl)-6-[(2-hydroxyethyl)(methylsulfonyl)amino]-N-methyl-1-benzofuran-3-carboxamide 10.0% granulation into #0E brown HPMC capsule shells with a target fill weight of 250 mg. Take four 20-capsule formulator samples from four equally divided time points during encapsulation.
22. Pass the capsules through a de-duster and inspect for any physical defects and correct capsule closure. Sort if necessary.
23. Store the finished capsules in sealed double polyethylene bags inside a rigid container at room temperature. Protect from light.

### EXAMPLE 2

### 200 MG Capsule

A 2.5 kg batch of a pharmaceutical formulation of 5-cyclopropyl-2-(4-fluorophenyl)-6-[(2-hydroxyethyl)(methylsulfonyl)amino]-N-methyl-1-benzofuran-3-carboxamide was made as follows:
1. Weigh the following ingredients—micronized 5-cyclopropyl-2-(4-fluorophenyl)-6-[(2-hydroxyethyl)(methylsulfonyl)amino]-N-methyl 1-benzofuran-3-carboxamide (1500.0 g), microcrystalline cellulose (Avicel PH101)(407.5 g), povidone (USP Plasdone K29/32)(100 g), sodium starch glycolate (NF)(75 g) and sodium lauryl sulfate (NF)(125 g).
2. Screen the ingredients from step 1 through a 20 mesh screen.
3. Add half of the microcrystalline cellulose into a suitable granulator. Then add micronized 5-cyclopropyl-2-(4-fluorophenyl)-6-[(2-hydroxyethyl)(methylsulfonyl)amino]-N-methyl-1-benzofuran-3-carboxamide, povidone, sodium starch glycolate and sodium lauryl sulfate into the granulator.
4. Add the other half of the microcrystalline cellulose into the granulator. Mix for 2 minutes with impeller on approximately 350 rpm. Determine density (approximately 0.4 g/ml).
5. Add 75 g polysorbate 80 to 300 g purified water with stirring for a minimum of 30 minutes (at low speed to minimize foam formation). Visually verify complete dissolution of the polysorbate 80.
6. Granulate the mix in step 4 with the solution from step 5 with impeller on at low speed and chopper off.
7. Add additional purified water if necessary and mix until granulation is complete.
8. After all granulating fluid is added, mix for an additional 1 minute with impeller on and chopper off. Record total amount of water used to granulate.
9. Dry the granulation in a fluid bed dryer with an inlet temperature of 50° C.±5° C. to a moisture content of about 1% to about 4% tested on a Computrac at 100° C. Note: Grind the granulation in a mortar before performing the moisture test. Weigh and record yield.
10. If two or more sub-batches of the granulation are made, add the sub-batches into a tumble type blender and blend for 5 minutes.
11. Perform a sieve analysis on the dried granulation with 42 mesh (0.0139 inch), 80 mesh (0.0070 inch), 150 mesh (0.0041 inch), 200 mesh (0.0029 inch), 325 mesh (0.0017 inch) and 400 mesh (0.0015 inch) sieves.
12. Pass the granulation through a cone mill approximately at 1000 rpm with 0.0394 inch round hole sieves.
13. Transfer the milled dried granulation from step 12 into a suitable tumble type blender (blending will be done without intensifier bar activation). Blend for 2 minutes. Note: Determine density (approximately 0.5 g/ml) and sieve analysis with same set of sieves. Take a 20 g formulator sample. Weigh and record yield. Review formulator particle size result before proceeding.
14. Store in a black double polyethylene bag in an appropriate container at room temperature until ready for final blend. Protect from light.
15. Based on the yield in step 13, calculate the amounts required for the dry addition. Theoretical amounts for a 2.5 kg batch are given as: silicon dioxide (colloidal, NF)(5 g), povidone (USP Plasdone K 29/32)(150 g), sodium starch glycolate (NF)(50 g) and magnesium stearate (NF/EP, vegetable grade)(12.5 g).
16. Transfer the milled dried granulation from step 14 into a tumble type blender.
17. Weigh silicon dioxide and pass through a 20 mesh screen and add into the tumble type blender. Blend for 5 minutes.
18. Weigh the povidone, sodium starch glycolate and pass through the 20 mesh screen and add into the tumble type dryer. Blend for 10 minutes.
19. Pass magnesium stearate through a 30 mesh screen and pre-mix with an approximately equal portion of blend (may bag blend for 15 sec), then add to the blend in the tumble type blender. Blend for 2 minutes. Note: Determine density (approximately 0.4 g/ml) and sieve analysis. Take 12 samples for blend uniformity (approximately 350 mg) and a 50 g formulator sample. Weigh and record yield. Store in a black double polyethylene bag in an appropriate container at room temperature until ready for encapsulation. Protect from light.
20. Set up a H&K capsule machine with parts for size #0E capsules (suggested dosing disk: 13.5 mm) and tamping pins for size #0E capsules.
21. Encapsulate the 5-cyclopropyl-2-(4-fluorophenyl)-6-[(2-hydroxyethyl)(methylsulfonyl)amino]-N-methyl-1-benzofuran-3-carboxamide 60.0% granulation into #0E brown HPMC capsule shells with a target fill weight of 333 mg. Take four 20-capsule formulator samples from four equally divided time points during encapsulation.
22. Pass the capsules through a de-duster and inspect for any physical defects and correct capsule closure. Sort if necessary.
23. Store the finished capsules in sealed double polyethylene bags inside a rigid container at room temperature. Protect from light.

### EXAMPLE 3

### 150 MG Tablet

A 150 mg tablet formulation of 5-cyclopropyl-2-(4-fluorophenyl)-6-[(2-hydroxyethyl)(methylsulfonyl)amino]-N-methyl-1-benzofuran-3-carboxamide was made to contain the following:

TABLE 1

| Ingredient | Amount (mg) | % weight |
|---|---|---|
| Micronized 5-cyclopropyl-2-(4- | 150 | 60 |
| fluorophenyl)-6-[(2- | | |
| hydroxyethyl)(methylsulfonyl)amino]- | | |
| N-methyl-1-benzofuran-3-carboxamide | | |
| Microcrystalline cellulose (Avicel | 40.75 | 16.3 |
| PH101) | | |
| Polysorbate 80, NF | 7.5 | 3 |
| Povidone, USP (Plasdone K 29/32) | 25 | 10 |
| Sodium starch glycolate, NF | 12.5 | 5 |
| Sodium lauryl sulphate, NF | 12.5 | 5 |
| Silicon dioxide, colloidal, NF | 0.5 | 0.2 |
| Magnesium stearate, NF/EP | 1.25 | 0.5 |
| Total | 250 | 100 |

The above-listed ingredients were wet granulated using a process similar to that in Examples 1 and 2 above. Then, the final blend was compressed on a Colton 204 Tablet Press equipped with 11/32 in standard concave, round punch. Tablets with target hardness ranging from 2-8 kp were made and tested for tablet weight, thickness, diameter, hardness, friability and dissolution. All tested parameters were within satisfactory limits.

### Bioavailability Testing

The pharmaceutical formulation of Example 2 was tested in fasted and fed dogs in a cross over fashion. Previously, 5-cyclopropyl-2-(4-fluorophenyl)-6-[(2-hydroxyethyl)(methylsulfonyl)amino]-N-methyl-1-benzofuran-3-carboxamide exhibited a 4.7-fold food effect when dosed in dogs from a 2% Tween 80/0.5% methylcellulose Tox suspension at a high dose of 300 mg/kg. The dose of Tween is also high at 100 mg/kg, which is not practical for human formulation. Another formulation containing 5% sodium lauryl sulfate was also used as a reference for simple dry blend formulation. The simple dry blend formulation exhibited 5.2-fold food effect consistent with the Tween suspension result. The pharmaceutical formulation of the present invention enhanced bioavailability about 3 times at fasted state and as a result reduced the food effect. The fed/fast ratio for the pharmaceutical formulation of the present invention is 1.1. The food effect study results are shown in Table 2 below.

TABLE 2

| | | | | 03127.001200 |
|---|---|---|---|---|
| | Dose | | AUC/Dose | Fed/Fasted |
| | (mg/kg) | | (ng hr/mL) | Ratio |
| Example 2 | 20.1 | mg/kg | 458 | 1.1 |
| | | Fasted | | |
| | 24.8 | mg/kg Fed | 505 | |
| Standard Dry | 29.0 | mg/kg | 172 | 5.2 |
| Blend Formulation | | Fasted | | |
| with 5% sodium | 24.7 | mg/kg Fed | 897 | |
| lauryl sulfate* | | | | |
| Tox Suspension | 300 | mg/kg | 22 | 4.7 |
| with 2% Tween 80 | | Fasted | | |
| and 0.5% Methyl | 300 | mg/kg Fed | 103 | |
| Cellulose** | | | | |

*dry blend also includes ProSolv SMCC 50, sodium starch glycolate and magnesium stearate;
**tox suspension also includes water

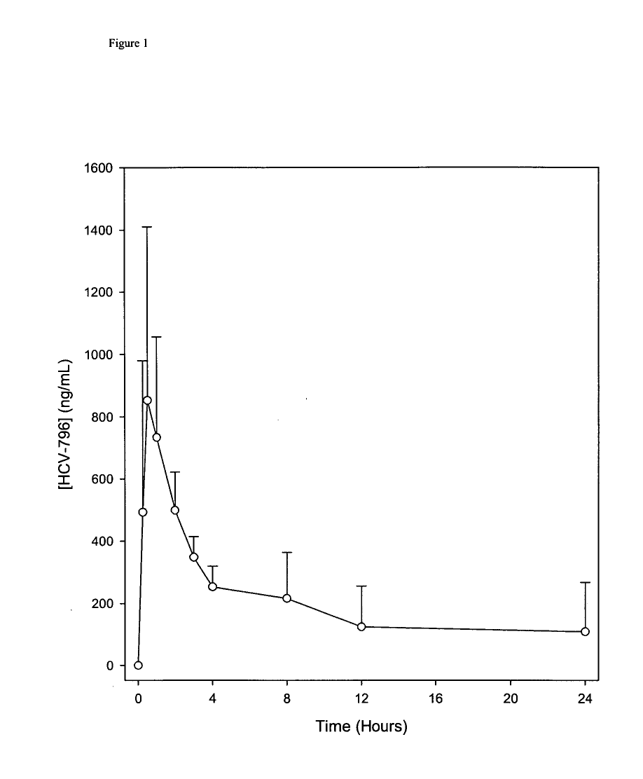

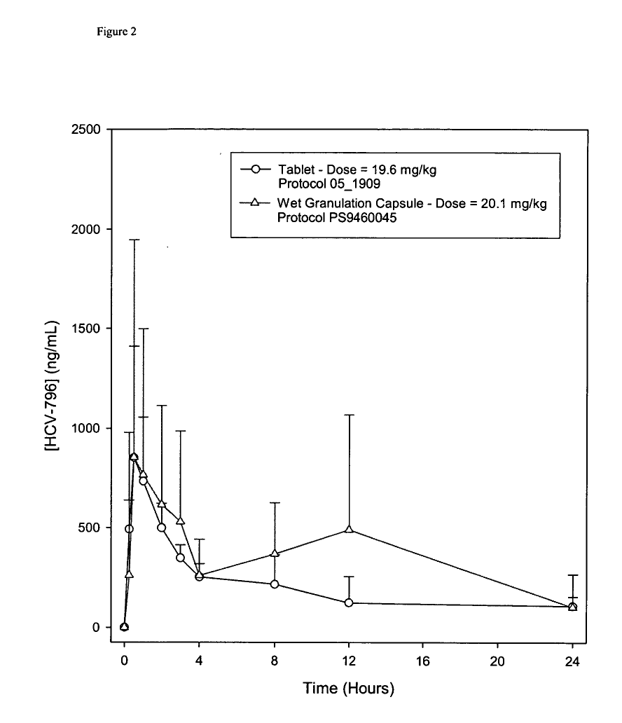

In addition, the pharmaceutical formulation of Example 3 was evaluated in four female Beagle dogs (7.0-8.8 kg). A single 150 mg dose (tablet) was administered to each dog following an overnight fast. Blood samples were drawn at 0 (predose), 0.25, 0.5, 1, 2, 3, 4, 8, 12 and 24 hours after dosing, plasma was separated and assayed for 5-cyclopropyl-2-(4-fluorophenyl)-6-[(2-hydroxyethyl)(methylsulfonyl)amino]-N-methyl-1-benzofuran-3-carboxamide content. The pharmacokinetic parameters were determined for each dog and descriptive statistics (AUC_{0-∞}, Cₘₐₓ, tₘₐₓ and t_{1/2}) were calculated. The results are summarized in Table 3 below and in FIGS. 1 and 2.

TABLE 3

| | |
|---|---|
| Dose | 21.4 |
| (mg/kg) | 20.8 |
| | 19.2 |
| | 17.0 |
| Mean | 19.6 |
| SD | 1.96 |
| AUC 0-∞ | 5099 |
| (ng · hr/mL) | 6959 |
| | 3912 |
| | 6761 |
| Mean | 5592 |
| SD | 1347 |
| AUC/Dose | 238 |
| | 317 |
| | 203 |
| | 397 |
| Mean | 289 |
| SD | 86.2 |
| Cₘₐₓ | 1534 |
| (ng/mL) | 900 |
| | 582 |
| | 807 |
| Mean | 956 |
| SD | 408 |
| Cₘₐₓ/Dose | 71.6 |
| | 43.2 |
| | 30.2 |
| | 47.4 |
| Mean | 48.1 |
| SD | 17.3 |
| tₘₐₓ | 0.50 |
| (hr) | 0.50 |
| | 2.00 |
| | 0.50 |
| Mean | 0.88 |
| SD | 0.75 |
| t_{1/2} | 8.41 |
| (hr) | 3.57 |
| | 5.71 |
| | 3.18 |
| Mean | 5.22 |
| SD | 2.40 |

In conclusion, 5-cyclopropyl-2-(4-fluorophenyl)-6-[(2-hydroxyethyl)(methylsulfonyl)amino]-N-methyl-1-benzofuran-3-carboxamide tablet formulation showed similar mean plasma level profiles to wet granulation capsule formulation (secondary peak in capsule profile due to one dog) at the comparable doses on a per kg basis administered (19.6 and 20.1 mg/kg, respectively). There was less variability observed with the tablet formulation relative to the wet granulation capsule, % CV's for AUC, 25% and 50%, respectively, and for Cₘₐₓ, 43% and 71%, respectively. The dose-normalized AUC from the tablet, 287 ng·hr/mL per mg/kg, was lower than that from the wet granulation capsule, 458 ng·hr/mL per mg/kg. However, the higher AUC from the wet granulation capsule was influenced by secondary peak in one dog. Excluding the dog with secondary peak, the dose-normalized AUC of the wet granulation capsule will be 363 ng·hr/mL per mg/kg.

### Stability Testing

The accelerated stability of the capsules of Examples 1 and 2 was studied. The capsules were packaged in HDPE bottles and stored at 40° C./75% RH and under ICH option 2 light condition. The samples were assayed by HPLC for potency and impurities and by dissolution apparatus. No apparent decrease in potency or increase in impurities was observed after 2 weeks under ICH option 2 light condition and after 3 months of storage at 40° C./75% RH for both the 25 mg and the 200 mg strengths. No change in dissolution was also observed under all conditions. The stability data for 5-cyclopropyl-2-(4-fluorophenyl)-6-[(2-hydroxyethyl)(methylsulfonyl)amino]-N-methyl-1-benzofuran-3-carboxamide pharmaceutical formulations of Examples 1 and 2 is shown in Table 4 below.

TABLE 4

| | | Strength | Impurity (%) | | | | Dissolution* |
|---|---|---|---|---|---|---|---|
| sample | Condition | (%) | RRT: 1.06 | RRT: 1.45 | RRT: 1.79 | RRT: 1.84 | % Released in 60 min. |
| 5-cyclopropyl-2-(4-fluorophenyl)-6-[(2- | Initial | 99.2 | <0.05 | 0.13 | 0.05 | 0.07 | NA |
| hydroxyethyl)(methylsulfonyl)amino]-N- | | | | | | | |
| methyl-1-benzofuran-3-carboxamide alone | | | | | | | |
| Example 1 | Initial | 102.1 | <0.05 | 0.14 | 0.05 | 0.08 | 101 |
| Example 1 | 26 d/ICH2 | 101.5 | 0.11 | 0.14 | 0.05 | 0.08 | 101 |
| Example 1 | 1 m/40 C./75% RH | 100.6 | 0.10 | 0.13 | 0.06 | 0.08 | 101 |
| Example 1 | 3 m/40 C./75% RH | TBD | TBD | TBD | TBD | TBD | TBD |
| Example 2 | Initial | 102 | 0.05 | 0.14 | 0.05 | 0.08 | 95 |
| Example 2 | 26 d/ICH2 | 99.8 | 0.07 | 0.13 | <0.05 | 0.08 | 96 |
| Example 2 | 1 m/40 C./75% RH | 99.7 | 0.06 | 0.13 | <0.05 | 0.08 | 96 |
| Example 2 | 3 m/40 C./75% RH | TBD | TBD | TBD | TBD | TBD | TBD |

*Dissolution method: 1% SLS in water, peddle 100 RPM followed by HPLC analysis.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A pharmaceutical formulation comprising:
a therapeutically effective amount of 5-cyclopropyl-2-(4-fluorophenyl)-6-[(2-hydroxyethyl)(methylsulfonyl)amino]-N-methyl-1-benzofuran-3-carboxamide; and
pharmaceutically acceptable additives, wherein said pharmaceutically acceptable additives comprise at least one surfactant.

2. The pharmaceutical formulation of claim 1, wherein the therapeutically effective amount of 5-cyclopropyl-2-(4-fluorophenyl)-6-[(2-hydroxyethyl)(methylsulfonyl)amino]-N-methyl-1-benzofuran-3-carboxamide ranges from about 1 mg to about 2000 mg.

3. The pharmaceutical formulation of claim 2, wherein the therapeutically effective amount of 5-cyclopropyl-2-(4-fluorophenyl)-6-[(2-hydroxyethyl)(methylsulfonyl)amino]-N-methyl-1-benzofuran-3-carboxamide ranges from about 25 mg to about 200 mg.

4. The pharmaceutical formulation of claim 1, wherein the 5-cyclopropyl-2-(4-fluorophenyl)-6-[(2-hydroxyethyl)(methylsulfonyl)amino]-N-methyl-1-benzofuran-3-carboxamide is micronized to a particle size specification of 50% less than or equal to 5 μm and 90% less than or equal to 20 μm.

5. The pharmaceutical formulation of claim 1, wherein the at least one surfactant is a blend of surfactants.

6. The pharmaceutical formulation of claim 5, wherein the blend of surfactants comprises sodium lauryl sulfate and polysorbate 80.

7. The pharmaceutical formulation of claim 6, wherein the sodium lauryl sulfate is present in an amount ranging from about 1% to about 10% by weight of the pharmaceutical formulation.

8. The pharmaceutical formulation of claim 6, wherein the polysorbate 80 is present in an amount ranging from about 1% to about 5% by weight of the pharmaceutical formulation.

9. The pharmaceutical formulation of claim 1, wherein the pharmaceutically acceptable additives further comprise at least one solubilizer.

10. The pharmaceutical formulation of claim 9, wherein the at least one solubilizer is povidone.

11. The pharmaceutical formulation of claim 10, wherein the povidone is present in an amount ranging from about 1% to about 20% by weight of the pharmaceutical formulation.

12. The pharmaceutical formulation of claim 1, wherein the pharmaceutically acceptable additives further comprise ingredients selected from the group consisting of diluents, surfactants, solubilizers, disintegrants, glidants, lubricants, colorants and combinations thereof.

13. The pharmaceutical formulation of claim 12, wherein the diluent is selected from microcrystalline cellulose, silicified microcrystalline cellulose, starches, mannitol, lactose, celluloses, calcium phosphates and combinations thereof.

14. The pharmaceutical formulation of claim 12, wherein the surfactant is selected from the group consisting of polysorbate 80, sodium lauryl sulfate, sugar esters of fatty acids, poloxamer, docusate sodium, polyoxyethylene sorbitan fatty acid esters, and combinations thereof.

15. The pharmaceutical formulation of claim 12, wherein the solubilizer is selected from the group consisting of povidone, poloxamer, glycerides of fatty acids, polyoxyethylene castor oil derivatives, and combinations thereof.

16. The pharmaceutical formulation of claim 12, wherein the disintegrant is selected from the group consisting of sodium starch glycolate, crospovidone, croscarmellose sodium, alginic acid, modified cellulose, pregelatinized starch, ion exchange resins, and combinations thereof.

17. The pharmaceutical formulation of claim 12, wherein the glidant is selected from the group consisting of colloidal silicon dioxide, talc, metal stearates, magnesium carbonate, calcium silicate, fumed silicon dioxide, and combinations thereof.

18. The pharmaceutical formulation of claim 12, wherein the lubricant is selected from the group consisting of magnesium stearate, metal stearates, glyceryl behenate, sodium stearyl fumarate, hydrogenated vegetable oils, fatty acids, and combinations thereof.

19. The pharmaceutical formulation of claim 1, wherein the pharmaceutical formulation takes the form of granulated 5-cyclopropyl-2-(4-fluorophenyl)-6-[(2-hydroxyethyl)(methylsulfonyl)amino]-N-methyl-1-benzofuran-3-carboxamide in a capsule.

20. The pharmaceutical formulation of claim 1, wherein the pharmaceutical formulation takes the form of a tablet.

21. A method of making a pharmaceutical formulation comprising the steps of:
(a) granulating 5-cyclopropyl-2-(4-fluorophenyl)-6-[(2-hydroxyethyl)(methylsulfonyl)amino]-N-methyl-1-benzofuran-3-carboxamide and pharmaceutically acceptable additives to form a granulate, wherein said pharmaceutically acceptable additives comprise at least one surfactant; and
(b) blending the granulate with pharmaceutically acceptable additives to form a final blend.

22. The method of claim 21, wherein the therapeutically effective amount of 5-cyclopropyl-2-(4-fluorophenyl)-6-[(2-hydroxyethyl)(methylsulfonyl)amino]-N-methyl-1-benzofuran-3-carboxamide ranges from about 1 mg to about 2000 mg.

23. The method of claim 22, wherein the therapeutically effective amount of 5-cyclopropyl-2-(4-fluorophenyl)-6-[(2-hydroxyethyl)(methylsulfonyl)amino]-N-methyl-1-benzofuran-3-carboxamide ranges from about 25 mg to about 200 mg.

24. The method of claim 21, wherein the 5-cyclopropyl-2-(4-fluorophenyl)-6-[(2-hydroxyethyl)(methylsulfonyl)amino]-N-methyl-1-benzofuran-3-carboxamide is micronized to a particle size specification of 50% less than or equal to 5 μm and 90% less than or equal to 20 μm.

25. The method of claim 21, wherein the at least one surfactant is a blend of surfactants.

26. The method of claim 20, wherein the blend of surfactants comprises sodium lauryl sulfate and polysorbate 80.

27. The method of claim 26, wherein the sodium lauryl sulfate is present in an amount ranging from about 1% to about 10% by weight of the pharmaceutical formulation.

28. The method of claim 26, wherein the polysorbate 80 is present in an amount ranging from about 1% to about 5% by weight of the pharmaceutical formulation.

29. The method of claim 21, wherein the pharmaceutically acceptable additives of step (a) further comprise at least one solubilizer.

30. The method of claim 29, wherein the at least one solubilizer is povidone.

31. The method of claim 30, wherein the povidone is present in an amount ranging from about 1% to about 20% by weight of the pharmaceutical formulation.

32. The method of claim 21, wherein the pharmaceutically acceptable additives further comprise ingredients selected from the group consisting of diluents, surfactants, solubilizers, disintegrants, glidants, lubricants, colorants and combinations thereof.

33. The method of claim 32, wherein step (a) comprises the steps of:
(a1) screening 5-cyclopropyl-2-(4-fluorophenyl)-6-[(2-hydroxyethyl)(methylsulfonyl)amino]-N-methyl-1-benzofuran-3-carboxamide, at least one diluent, at least one solubilizer, at least one disintegrant, and at least one surfactant into a granulator to form a screened material;
(a2) blending the screened material to form a screened/blended material;
(a3) dissolving at least one surfactant in water to form a surfactant solution;
(a4) granulating the screened/blended material with the surfactant solution to form a wet granulation;
(a5) drying the wet granulation to form a dried granulation; and
(a6) milling the dried granulation to form the granulate.

34. The method of claim 33 further comprising the step of:
(a4*) adding additional water to facilitate granulation.

35. The method of claim 32, wherein step (b) comprises the steps of:
(b1) blending at least one screened glidant with the granulate from step (a) to form a first blend;
(b2) blending the first blend with at least one screened solubilizer and at least one screened disintegrant to form a second blend;
(b3) blending a portion of the second blend with an equal amount of at least one screened lubricant to form a third blend; and
(b4) blending the third blend with the remaining second blend to form the final blend.

36. The method of claim 21 further comprising the step of:
(c) encapsulating the final blend to form the pharmaceutical formulation.

37. The method of claim 21 further comprising the step of:
(c) compressing the final blend to form the pharmaceutical formulation in the form of a tablet.

38. A pharmaceutical formulation made according to the method of claim 21.

39. A method of inhibiting hepatitis C virus, wherein the method comprises administering a pharmaceutical formulation as defined in claim 1 to a subject in need of such treatment.
